(19) United States
(12) Patent Application Publication
(10) Pub. No.: US 2012/0238520 A1
(43) Pub. Date: Sep. 20, 2012

(54) Novel Medical Use

(76) Inventor: David Lipson, King of Prussia, PA (US)

(21) Appl. No.: 13/512,901

(22) PCT Filed: Dec. 3, 2010
(86) PCT No.: PCT/EP2010/068864

§ 371 Date: May 31, 2012

Related U.S. Application Data
(60) Provisional application No. 61/267,909, filed on Dec. 9, 2009.

(51) Int. Cl. A61K 31/7076; A61P 9/10; A61P 11/00; C07H 19/16; A61P 29/00

(52) U.S. Cl. 514/46; 536/27.21

(57) ABSTRACT

The present invention relates to a compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol

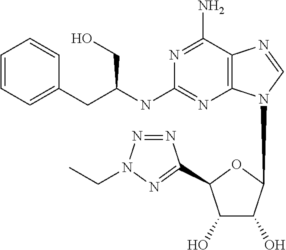

or a pharmaceutically acceptable salt thereof for use in the treatment or prevention of inflammatory and non-inflammatory diseases abd conditions associated with alveolar filling.

The present invention relates to the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation, with a compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof.

Acute respiratory distress syndrome (ARDS) is a form of severe acute lung injury (ALI) characterized by hypoxemic respiratory failure and non-cardiogenic pulmonary edema. [Bernard G R, Artigas A, Brigham K L, Carlet J, Falke K, Hudson L, Lamy M, Legall J R, Morris A and Spragg R; The American-European Consensus Conference on ARDS. Definitions, mechanisms, relevant outcomes, and clinical trial coordination; Am. J. Respir. Crit. Care Med. 1994 March; 149(3 Pt 1): 818-824]. The syndrome may be caused by either direct or indirect insult to the lung; including, severe trauma, multiple blood transfusions, sepsis, pneumonia, burn injury, pancreatitis, or ischemia-reperfusion injury. It is observed in both medical and surgical patients as a final common pathway from many types of injury and is associated with mortality that may approach 40-50%. [The Acute Respiratory Distress Syndrome Network, N. Engl. J. Med. 2000, 342: 1301-1308; and Rubenfeld G D, Caldwell E, Peabody E, et al., N. Engl. J. Med. 2005, 353: 1685-1693]. Mortality appears to be highest in patients with advanced age, and higher organ dysfunction and lung injury scores. Patients often die from the development of multiple organ dysfunction syndrome (MODS). There are believed to be no established pharmacologic therapies for this serious syndrome. Regardless of the cause of injury, ALI and ARDS pathologically are characterized by diffuse alveolar damage (DAD). Lung biopsies reveal inflammation and pulmonary edema, hyaline membrane formation, and type II pneumocyte hyperplasia. Injury to the lung endothelium causes increased capillary permeability and a movement of protein-rich fluid into the pulmonary alveolar space.

A primary mechanism involved in driving alveolar fluid clearance in the lung is the active transport of sodium and chloride across the alveolar epithelium. Factor [Factor P, Mutlu G M, Chen L, et al., Proc. Natl. Acad. Sci. USA, 2007, 104: 4083-4088; and Kreindler J L and Shapiro S D, Nature Medicine, 2007, 13: 406-408] suggested through in vivo and in vitro mouse experiments that A2a receptors may play a pivotal role in regulating alveolar fluid clearance. Thus adenosine analogues, shown to help decrease alveolar edema, would be useful in pathologic states associated with pulmonary edema.

There are currently no approved pharmacologic treatments for acute lung injury; there is thus a perceived unmet need for therapeutic agents to treat patients with acute lung injury.

The present invention provides a compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol:

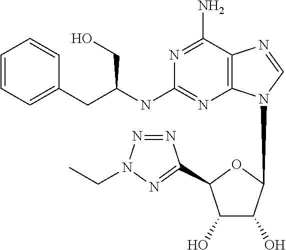

or a pharmaceutically acceptable salt thereof for use in the treatment or prevention of inflammatory and non-inflammatory diseases and conditions associated with alveolar filling.

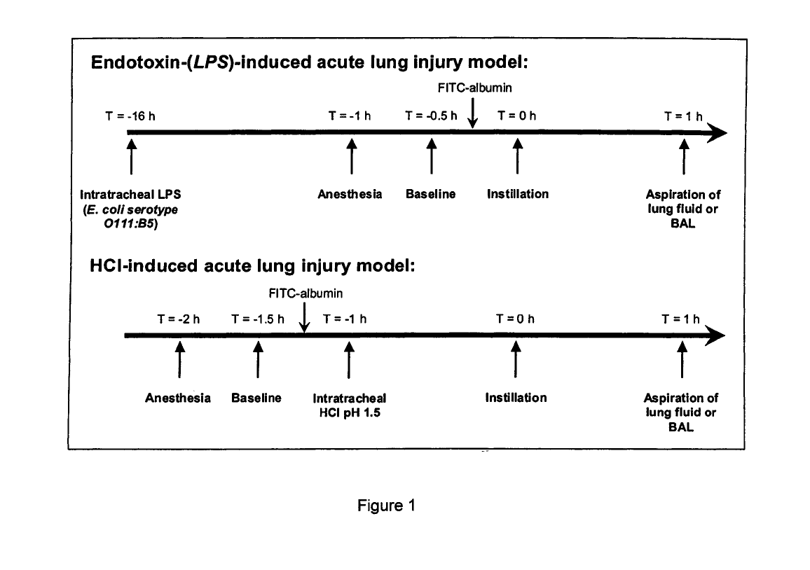

FIG. 1 shows time schemes for the two models of acute lung injury used, namely endotoxin (LPS) and acid aspiration (HCl).

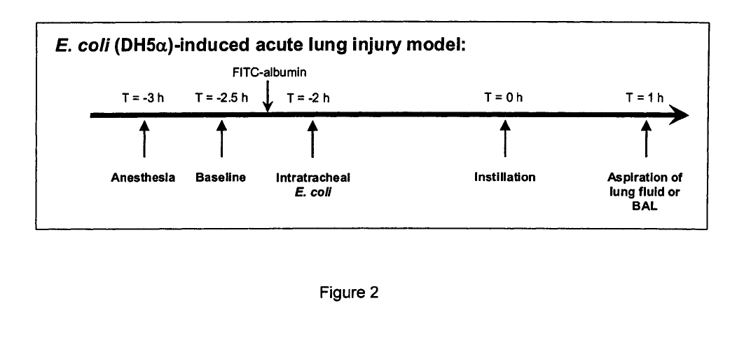

FIG. 2 shows the time scheme for the E. coli (DH5α) model of acute lung injury.

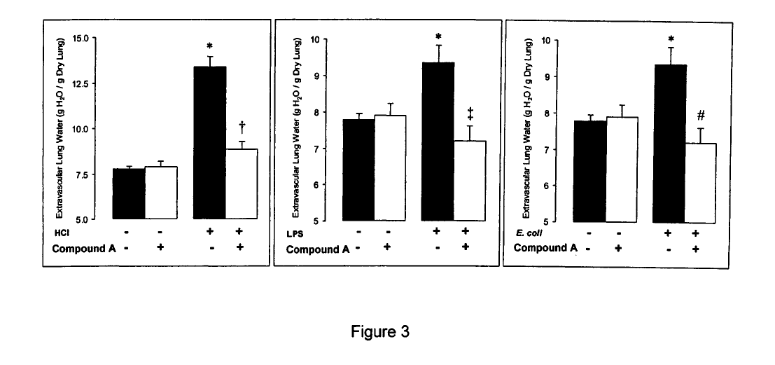

FIG. 3 shows edema formation measured as extravascular lung water in rats 2 h after intratracheal HCl, pH 1.5, instillation (Chemical injury), 16 h after intratracheal LPS, E. coli serotype O111:B5 (5 mg/kg body wt), instillation (Biochemical injury), and 3 h after intratracheal E. coli DH5α instillation (Biological injury) with and without 10⁻⁴ M Compound A. *P<0.05 compared to Control; †P<0.05 compared to HCl; ‡P<0.05 compared to LPS; #P<0.05 compared to E. coli; one-way ANOVA with Tukey's test post hoc.

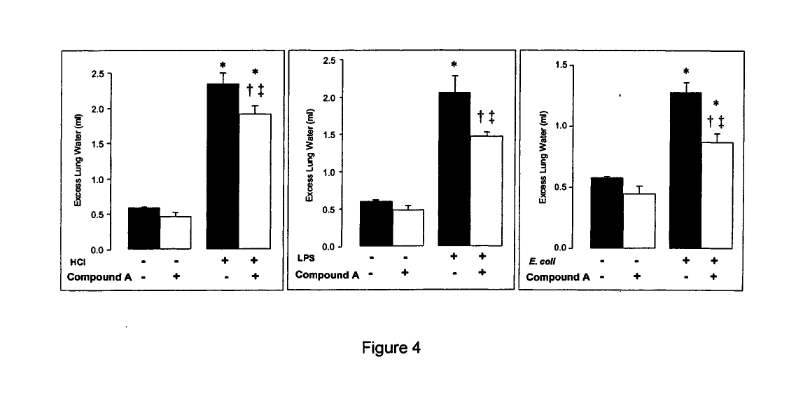

FIG. 4 shows edema formation measured as excess lung water in rats 2 h after intratracheal HCl, pH 1.5, instillation (Chemical injury), 16 h after intratracheal LPS, E. coli serotype 0111:B5 (5 mg/kg body wt), instillation (Biochemical injury), and 3 h after intratracheal E. coli DH5α instillation (Biological injury) with and without 10⁻⁴ M Compound A. *P<0.05 compared to Control; †P<0.05 compared to HCl, LPS, or E. coli in respective figure; ‡P<0.05 compared to Compound A; one-way ANOVA with Tukey's test post hoc.

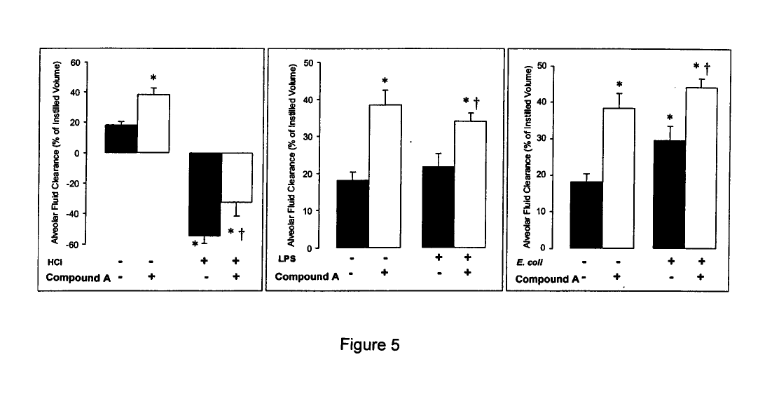

FIG. 5 shows alveolar fluid clearance in rats 2 h after intratracheal HCl, pH 1.5, instillation (Chemical injury), 16 h after intratracheal LPS, E. coli serotype 0111:B5 (5 mg/kg body wt), instillation (Biochemical injury), and 3 h after intratracheal E. coli DH5α instillation (Biological injury) with and without 10⁻⁴ M Compound A. *P<0.05 compared to Control; †P<0.05 compared to HCl, LPS, or E. coli in respective figure; †P<0.05 compared to Compound A; one-way ANOVA with Tukey's test post hoc.

In each of FIGS. 3, 4 and 5, Compound A is (2R,3R,4S,5R)-2-(6-amino-2-{[(1S)-2-hydroxy-1-(phenyl methyl)ethyl]amino}-9H-purin-9-yl)-5-(2-ethyl-2H-tetrazol-5-yl)tetrahydro-3,4-furandiol maleate.

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol, as a potent A2a agonist and A3 antagonist, is believed to have anti-inflammatory effects on cells (i.e. neutrophils) associated with the development of acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation. The combined anti-inflammatory effects as well as utility in decreasing pulmonary edema means (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol may be useful for treating and preventing diseases and conditions associated with pulmonary edema and inflammation.

Inflammatory and non-inflammatory diseases and conditions associated with alveolar filling include but are not limited to:

ALI following Cardio-Pulmonary Bypass

Lung protective solution containing (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may be dosed initially to patients at high risk of lung injury following bypass to prevent ALI. It is potentially useful in all patients on cardiopulmonary bypass (i.e. CABG with bypass) in order to decrease development of acute lung injury in this high-risk population.

### Ischemia-Reperfusion Injury

The isolated lung may be preserved in a solution containing (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof in order to decrease the risk of lung ischemia-reperfusion injury.

### Alveolar Edema of Donor Lungs

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may be added to organ preservation solution in order to increase the number of usable solid organ donor lungs by decreasing alveolar edema and improving the oxygenation index in a potential brain-dead organ donor.

### Non-Cardiogenic Pulmonary Edema

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may also be useful for the treatment or prevention of non-cardiogenic pulmonary edema caused by drug reaction, Transfusion Related Acute Lung Injury (TRALI), ALI, ARDS, Acute Interstitial pneumonia (AIP) and/or Hypersensitivity Pneumonitis.

### Primary Graft Failure (PGF)

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof administered to a brain-dead donor or to an organ recipient at time of transplantation may decrease risk of development of primary graft failure (acute lung injury of allograft, usually seen within 72 hours of transplantation).

In addition, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may also be useful during diuresis because Ata agonist/A3 antagonists augment alveolar clearance. Thus, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may be nephro-protective. The compound added to Renal-Protective Solution may enable less diuretic, which may be nephrotoxic, to be given leading to a preservation of renal function.

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may be in crystalline or amorphous form. Furthermore, this compound or pharmaceutically acceptable salt thereof may exist in one or more polymorphic forms. Thus, the present invention includes within its scope the use of all polymorphic forms of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof.

It will be appreciated that many organic compounds can form solvates with the solvents in which they are reacted or from which they are precipitated or crystallized. For example, a solvate with water is known as a “hydrate”. Solvents with high boiling points and/or solvents with a high propensity to form hydrogen bonds such as water, xylene, N-methylpyrrolidinone and methanol may be used to form solvates. Thus, the use of solvates of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof are within the scope of the invention. (2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol may be in the form of and may be administered as a pharmaceutically acceptable salt. For a review on suitable salts see Berge et al., J. Pharm. Sci., 1977, 66, 1-19. Suitable pharmaceutically acceptable salts include acid and base addition salts.

A pharmaceutically acceptable acid addition salt can be formed by reaction of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol with a suitable inorganic or organic acid (such as hydrobromic, hydrochloric, formic, sulfuric, nitric, phosphoric, succinic, maleic, acetic, fumaric, citric, tartaric, benzoic, p-toluenesulfonic, methanesulfonic, naphthalenedisulfonic acid (e.g. 1,5-naphthalenedisulfonic acid) or naphthalenesulfonic acid), optionally in a suitable solvent such as an organic solvent, to give the salt which is usually isolated for example by crystallisation and filtration. Thus, a pharmaceutically acceptable acid addition salt of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol can be for example a hydrobromide, hydrochloride, formate, sulfate, nitrate, phosphate, succinate, maleate, acetate, fumarate, citrate, tartrate, benzoate, p-toluenesulfonate, methanesulfonate, naphthalenedisulfonate (e.g. 1,5-naphthalenedisulfonate) or naphthalenesulfonate salt.

In an embodiment, the invention provides the use of the maleate salt of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol. In a further embodiment, the invention provides the use of the monohydrochloride salt of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol.

Included within the scope of the invention is the use of any and all solvates e.g. hydrates and polymorphs of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or pharmaceutically acceptable salts thereof in the treatment or prevention of inflammatory and non-inflammatory diseases associated with alveolar filling. In an embodiment, the invention provides the use of a solvate of the maleate salt of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol in the treatment or prevention of inflammatory and non-inflammatory diseases associated with alveolar filling. In a further embodiment, the invention provides the use of a hydrate of the maleate salt of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol in the treatment or prevention of inflammatory and non-inflammatory diseases associated with alveolar filling.

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may be prepared according to the general methods and experimental section provided in International Patent Application Publication Number WO1998/28319 (U.S. Pat. Nos. 6,426,337 and 6,528,494) (see in particular Example 11, especially, Example 11a and 11e referred to as (2R,3R,4S,5R)-2-[6-amino-2-(1S-hydroxymethyl-2-phenyl-ethylamino)-purin-9-yl]-5-(2-ethyl-2H-tetrazol-5-yl)-tetrahydro-furan-3,4-diol).

(2R,3R,4S,5R)-2-(6-Amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof is expected to have beneficial anti-inflammatory effects and therefore may be of use in the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

It will be appreciated by those skilled in the art that references herein to treatment or therapy may extend to prophylaxis as well as the treatment of established conditions. Treatment would be defined as institution of therapy after the disease has developed. Prevention, or prophylaxis, would be defined as administration of the compound in a patient at risk for development of ALI or ARDS in order to prevent or mitigate the severity of the disease in this patient. In an embodiment, the invention provides the treatment of established conditions.

According to the first aspect of the invention there is provided (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) for use in the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

According to the second aspect of the invention there is provided the use of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) in the manufacture of a medicament for the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

In the third aspect of the invention, there is provided a method for the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation, to a human in need thereof, which method comprises administering an effective amount of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt).

When used in therapy, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) may be formulated in a suitable pharmaceutical composition. Such pharmaceutical compositions can be prepared using standard procedures.

Thus, in the fourth aspect the invention provides a pharmaceutical composition which comprises a compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) optionally with one or more pharmaceutically acceptable carriers and/or excipients for use in the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

In the fifth aspect the invention provides the use of a pharmaceutical composition which comprises a compound which is (2R,3R,4S,5R)-2-(6-amino-2-{[(1S)-2-hydroxy-1-(phenyl methyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) optionally with one or more pharmaceutically acceptable carriers and/or excipients for the manufacture of a medicament for the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

In the sixth aspect the invention provides a method for the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation, to a human in need thereof, which method comprises administering an effective amount of a pharmaceutical composition comprising (2R,3R,4S,5R)-2-(6-amino-2-{[(1S)-2-hydroxy-1-(phenyl methyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) with one or more pharmaceutically acceptable carriers and/or excipients.

In the seventh aspect the invention provides a pharmaceutical composition for use in the treatment or prevention of inflammatory and non-inflammatory diseases and conditions associated with alveolar filling, in particular acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation, which comprises a compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof (e.g. maleate or monohydrochloride salt) optionally with one or more pharmaceutically acceptable carriers and/or excipients.

A pharmaceutical composition comprising (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof, may be adapted for oral, parenteral, rectal or inhaled administration and, as such, may be in the form of tablets, capsules, liquid preparations e.g. oral liquid preparations, powders, granules, lozenges, reconstitutable powders, injectable or infusible solutions or suspensions or suppositories. Suitable compositions may be prepared according to methods well known in the art for each particular type of composition.

In another aspect of the invention, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof is adapted for parenteral administration, i.e. intravenous administration. In another aspect of the invention, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof is adapted for administration to a patient by inhalation.

The compositions may contain from about 0.1% to 99% by weight, such as from about 10 to 60% by weight, of the active material, depending on the method of administration. The dose of the compound used in the treatment of the aforementioned disorders will vary in the usual way with the nature and seriousness of the disorders, the weight of the sufferer, and other similar factors. However, as a general guide suitable unit doses may be about 0.05 to 1000 mg, more suitably about 1.0 to 200 mg, for example 20 to 100 mg, and such unit doses may be administered more than once a day, for example two or three a day. Such therapy may extend for a number of days, weeks or months. In one embodiment compounds and pharmaceutical compositions according to the invention are suitable for inhaled or parenteral administration and/or are capable of once daily administration, for example at a dose in the range of 20 to 200 mg (e.g. about 20 to 100 mg, such as about 10 to 50 mg).

Generally, compositions suitable for inhaled administration may conveniently be formulated as aerosols, solutions, suspensions, drops, gels or dry powders, optionally with one or more pharmaceutically acceptable carriers and/or excipients such as aqueous or non-aqueous vehicles, thickening agents, isotonicity adjusting agents, antioxidants and/or preservatives.

For patients who are incapacitated, suspensions and solutions comprising (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof may also be conveniently administered via a nebulizer. Thus, in an embodiment, (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or a pharmaceutically acceptable salt thereof is adapted for administration via a nebulizer.

The solvent or suspension agent utilized for nebulization may be any pharmaceutically acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### Biological Data

### Methods

The preparation of anesthetized, ventilated rats was used to carry out the below described examples. Techniques of the below described examples have been published (Frank J A, Gutierrez J A, Jones K D, et al.: Low tidal volume reduces epithelial and endothelial injury in acid-injured rat lungs. Am. J. Respir. Crit. Care Med. 165:242-249, 2002; Folkesson H G, Nitenberg G, Oliver B L, et al.: Upregulation of alveolar epithelial fluid transport after subacute lung injury in rats from bleomycin. Am. J. Physiol. 275:L478-490, 1998; Folkesson H G, Norlin A, Wang Y, et al., Dexamethasone and thyroid hormone pretreatment upregulate alveolar epithelial fluid clearance in adult rats. J. Appl. Physiol. 88: 416-424, 2000; and Norlin A, Lu L N, Guggino S E, et al., Contribution of amiloride-insensitive pathways to alveolar fluid clearance in adult rats. J. Appl. Physiol. 90: 1489-1496, 2001).

### Pathological Examples:

The methods used are published in the above given references. A test solution of 5% albumin with and without 10′ M (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate dissolved in 0.9% NaCl was used and instilled into the airspaces of rats to measure alveolar fluid clearance with and without the injury-inducing agent (HCl, LPS, or E. coil, see below for details). Prior to test solution instillation, FITC-labeled albumin was injected to measure bi-directional protein movement across the lung endothelium and the epithelial barriers of the lung (FIGS. 1 and 2). Studies were carried out over 1-2 h to evaluate the effects of the adenosine A₂ agonist (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate on lung fluid balance during these two pathological conditions.

### HCl Examples:

To evaluate the effect of HCl (chemical injury; a model of aspiration-induced acute lung injury), HCl at pH 1.5 was used (FIG. 1, bottom panel illustrates the time scheme used for HCl treatments). Rats were surgically prepared (anesthetized, tracheotomized, outfitted with a carotid artery catheter, and connected to a ventilator). The rats were ventilated for a 30 min baseline of stable blood gases, the vascular tracer (FITC-labeled albumin) was given after 15 min stabilization, and HCl, pH 1.5, was then instilled into the distal airspaces of the lung after the 30-min baseline had passed. The injury was then allowed to develop for 1 h. Blood was sampled for fluorescence and arterial blood gas measurements every 30 min throughout the experiment. After 1 h, the test solution (5% albumin with and without (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was instilled to cover the same area of the lung as the acid and the rats were followed for an additional 1 h. The rats were then euthanized and samples (blood, alveolar edema fluid, lung tissue) were obtained. Alveolar fluid clearance, extravascular lung water, and endothelial-epithelial barrier leak were determined.

### LPS Examples:

To evaluate the effect of endotoxin (biochemical injury; a model of sepsis-induced acute lung injury), E. coli LPS at the concentration of 5 mg/kg body wt was given intratracheally (FIG. 1, top panel illustrates the time scheme used for LPS treatments). Rats were under a brief isofluorane anesthesia instilled intratracheally with 5 mg/kg body wt LPS (from E. coli serotype 0111:85) in 1 ml/kg body wt 0.9% NaCl. Sixteen hours later, the rats were surgically prepared (anesthetized, tracheotomized, outfitted with a carotid artery catheter, and connected to a ventilator). The rats were ventilated for a 30 min baseline of stable blood gases, the vascular tracer (FITC-labeled albumin) was given after 15 min stabilization, and the 5% albumin solution with and without (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was instilled into the distal airspaces of the lung. Blood was sampled for fluorescence and arterial blood gas measurements every 30 min throughout the experiment. After 1 h the rats were euthanized and samples (blood, alveolar edema fluid, lung tissue) were obtained. Alveolar fluid clearance, extravascular lung water, and endothelial-epithelial barrier leak were determined.

E. coli Examples:

To evaluate the effect of live E. coli (biological injury; a model of sepsis-induced acute lung injury), E. coli (serotype DH5α at the concentration of 10⁸ cfu/ml was given intratracheally (FIG. 2). Rats were surgically prepared (anesthetized, tracheotomized, outfitted with a carotid artery catheter, and connected to a ventilator). The rats were ventilated for a 30 min baseline of stable blood gases. An instillation catheter was passed to rest just above the bronchial carina and 1 ml/kg body wt of the solution containing 10⁸ cfu/ml E. coli DH5α was rapidly instilled into the lungs. The vascular tracer (FITC-labeled albumin) was given after 15 min stabilization time and the 5% albumin solution with and without (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was instilled 2 h later into the distal airspaces of the lung. Blood was sampled for fluorescence and arterial blood gas measurements every 30 min throughout the experiment. After a total of 3 h after the live E. coli instillation, the rats were euthanized and samples (blood, alveolar edema fluid, lung tissue) were obtained. Alveolar fluid clearance, extravascular lung water, and endothelial-epithelial barrier leak were determined.

### Pulmonary Edema Formation.

There was an increase in EVLW 2 h after HCl instillation (HCl: N=8; HCl+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=8) that suggested pulmonary edema (FIG. 3, left). (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate by itself was believed not to result in edema formation measured as increased EVLW in any experimental group. However, when (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was administered to the rats that 2 h earlier received HCl, a reduction in EVLW was observed, albeit EVLW remained elevated in those rats (FIG. 3, left).

In LPS-instilled rats, there was also observed an increase in EVLW 16 h after LPS instillation (LPS: N=7; LPS+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=6) (FIG. 3, center). As (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was administered to the rats that 16 h earlier received LPS, a reduction in EVLW was observed (FIG. 3, center).

In live E. coli-instilled rats. there was again an observed increase in EVLW 3 h after live E. coli instillation (E. coli: N=6; E. coli+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=6) (FIG. 3, right). As (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate was administered to the rats that 3 h earlier received live E. coli, a reduction in EVLW was observed (FIG. 3, right).

There was an observed increase in ELW 2 h after HCl instillation (HCl: N=8; HCl+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=8) (FIG. 4, left) that supported the previously measured increase in EVLW (FIG. 3, left). In contrast, when the rats that were given HCl 2 h earlier were instilled with (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate, ELW was reduced by ˜20% (FIG. 4, left). ELW remained, however, elevated compared to both control and (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate.

ELW was also determined in the rat lungs 16 h after LPS was instilled. There was again an observed increase in ELW 16 h after LPS instillation (LPS: N=7; LPS+also determined: N=6) (FIG. 4, center) that supported the previously measured increase in EVLW (FIG. 3, center). In contrast, when the rats that were given LPS 16 h earlier were instilled with (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate, ELW was reduced by ˜40% (FIG. 4, center).

ELW was then evaluated in the rat lungs 3 h after live E. coli was instilled. There was an observed increase in ELW 3 h after live E. coli instillation (E. coli: N=6; E. coli+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=6) (FIG. 4, right) that supported the previously measured increase in EVLW (FIG. 3, right). In contrast, when the rats that were given live E. coli 3 h earlier were instilled with (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate, ELW was reduced by ˜40% (FIG. 4, right).

### Alveolar Fluid Clearance.

Instillation of HCl 2 h earlier completely reduced fluid clearance and appeared to induce secretion of fluid into the airspaces (negative alveolar fluid clearance) (FIG. 5, left). (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate appeared to decrease the rate of alveolar fluid secretion (leak) in the HCl instilled rats in spite of the lung injury (FIG. 5, left). (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate may have been able to do so due to its protective effects on edema formation, vascular leak, and epithelial barrier leak.

In order to examine the effect of LPS on alveolar fluid clearance, LPS-instilled rats were evaluated with or without the addition of (2R,3R,4S,5R)-2-(6-amino-2-{[(1S)-2-hydroxy-1-(phenyl methyl)ethyl]amino}-9H-purin-9-yl)-5-(2-ethyl-2H-tetrazol-5-yl)tetrahydro-3,4-furandiol maleate (LPS: N=7; LPS+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=7). Instillation of LPS 16 h earlier appeared not to affect alveolar fluid clearance (FIG. 5, center). However, the ability of (2R,3R,4S,5R)-2-(6-amino-2-{[(1S)-2-hydroxy-1-(phenyl methyl)ethyl]amino}-9H-purin-9-yl)-5-(2-ethyl-2H-tetrazol-5-yl)tetrahydro-3,4-furandiol maleate to increase the rate of alveolar fluid clearance in these LPS instilled rats appeared to be retained in spite of the lung injury (FIG. 5, center).

In order to examine the effect of live E. coli on alveolar fluid clearance, live E. coli-instilled rats were evaluated with or without the addition of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate (E. coli: N=6; E. coli+(2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate: N=6). Instillation of live E. coli 3 h earlier appeared to stimulate alveolar fluid clearance (FIG. 5, right), a phenomenon observed in earlier experimental studies (Matthay, Physiol Rev 2002). The ability of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol maleate to increase the rate of alveolar fluid clearance in these live E. coli instilled rats appeared also to be retained in spite of the lung injury (FIG. 5, right).

What is claimed is:

1. A compound which is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol: 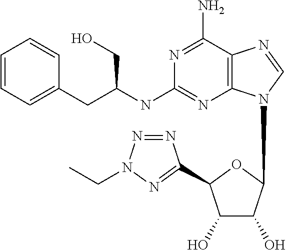
or a pharmaceutically acceptable salt thereof for use in the treatment or prevention of inflammatory and non-inflammatory diseases and conditions associated with alveolar filling.

2. The compound for use according to claim 1, wherein the compound is in the form of a maleate salt.

3. The compound for use according to claim 1, wherein the compound is in the form of a monohydrochloride salt.

4. The compound for use according to claim 1, wherein the inflammatory and non-inflammatory disease is acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation.

5. The compound for use according to claim 1 wherein the compound is adapted for parenteral administration or administration to a patient by inhalation.

6-11. (canceled)

11. A method for the treatment or prevention of inflammatory and non-inflammatory diseases and conditions associated with alveolar filling, to a human in need thereof, which method comprises administering an effective amount of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol 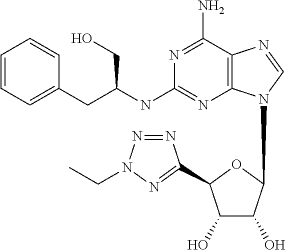
or a pharmaceutically acceptable salt thereof.

12. A method according to claim 11, wherein the compound is in the form of a maleate salt.

13. A method according to claim 11, wherein the compound is in the form of a monohydrochloride salt.

14. A method for the treatment or prevention of acute lung injury (including ALI following cardio-pulmonary bypass), acute respiratory distress syndrome, ischemia-reperfusion injury, alveolar edema of donor lungs, non-cardiogenic pulmonary edema and primary graft failure following lung transplantation, according to claim 11.

15. A method according to claim 11 wherein the compound is adapted for parenteral administration or administration to a patient by inhalation.
